(19) United States
(12) Patent Application Publication
(10) Pub. No.: US 2017/0080135 A1
(43) Pub. Date: Mar. 23, 2017

(54) Tee Joint Component and Breast Pump with Same

(71) Applicant: Junbo Chen, Shantou, Guangdong (CN)

(72) Inventor: Junbo Chen, Shantou, Guangdong (CN)

(21) Appl. No.: 15/021,949

(22) PCT Filed: Sep. 29, 2015
(86) PCT No.: PCT/CN2015/000670

§ 371 Date: Mar. 15, 2016

(30) Foreign Application Priority Data
May 5, 2015 (CN) 201510222792.1

(51) Int. Cl. A61M 1/06

(52) U.S. Cl. CPC A61M 1/062; A61M 1/06

(57) ABSTRACT

The present invention relates the field of mother and baby products, in particular to a breast pump and components thereof. The tee joint component is different from others in that the tee joint component is only provided with one negative pressure input interface; a milking shield and a milk outflow end disposed on the tee joint component are both integrated with the body of the tee joint component; the milk outflow end is a V-shaped one-way valve; the whole tee joint component is made of a flexible material. The cross-sectional thickness of both ends is greater than that of the middle region of the tee joint component. The flexible material is food-grade silicone rubber.

### TECHNICAL FIELD

The present invention relates to a mother and baby product, in particular to a breast pump and components thereof.

### BACKGROUND ART

As a mother and baby product, a breast pump is divided into a manual breast pump and an electric breast pump in the prior art. A breast pump usually consists of a milking shield fitting a female's breast, a bottle for collecting milk, a negative pressure generating means and a tee joint component connecting these three parts. Usually a tee joint component comprises three couplings: a milking shield coupling, a cylinder coupling and a bottle coupling. The milking shield coupling is provided with a trumpet-shaped milking shield; the cylinder coupling is provided with a cylinder head, and the inner surface of the cylinder coupling is attached with cylinder silica gel; the bottle coupling is connected to the bottle.

Tee joint components in the prior art have the following defects: 1, the tee joint components only serve for connection, and do not have other functions; 2, too many joints may cause improperly assembly, which will lead to air leak, thus affecting the milking effect. The most important parameter to measure the pros and cons of a breast pump is the magnitude of negative pressure suction, while in the prior art, all approaches to increase the magnitude of the negative pressure suction are related to the source, i.e. the negative pressure generating means; however, the negative pressure loss during transmission is ignored; 3, the tee joint components are difficult to clean and inconvenient to disassemble and assemble; 4, during the milking process, the parts exposed to milk are all made of food-grade silicone rubber, thereby making milk pure to the most extent.

### SUMMARY OF THE INVENTION

The present invention aims to overcome the defects in the prior art and to provide a tee joint component and a breast pump with the same. Compared with the prior art, the tee joint component can reduce the negative pressure loss during transmission, thereby enhancing breast pump suction; the tee joint component is very convenient to clean, disassemble and assemble; and meanwhile, with the use of a single passage, the purity of milk is ensured; the sanitary conditions of the breast pump are improved and the sterilization process is also simplified.

In order to achieve the above goals, the following specific technical solutions are taken:

The first technical solution:

A tee joint component is provided, wherein the tee joint component is only provided with one negative pressure input interface; a milking shield and a milk outflow end disposed on the tee joint component are both integrated with the body of the tee joint component; the milk outflow end is a V-shaped one-way valve; the whole tee joint component is made of flexible materials. The tee joint component is characterized in that the tee joint component is only provided with one negative pressure input interface; the milking shield and the milk outflow end disposed on the tee joint component are both integrated with the body of the tee joint component; the milk outflow end is a V-shaped one-way valve; the whole tee joint component is made of flexible materials.

The cross-sectional thickness of both ends is greater than that of the middle region of the tee joint component; specifically, the cross-sectional thickness of the first region and the third region is greater than that of the second region and the fourth region.

The flexible material is food-grade silicone rubber.

The second technical solution:

A breast pump is provided, comprising a negative pressure pump and a bottle, and further comprising the tee joint component of the first technical solution, connected with the negative pressure pump and the bottle.

The tee joint component is embedded in a support. The support frame is connected with a tee joint adapter. The tee joint adapter is connected with the bottle.

Unlike the traditional tee joint components made of plastics or other rigid materials, the tee joint component is overall made of food-grade silicone rubber, which can effectively compress the space of a negative pressure passage, simplify the part assembly process, and reduce the negative pressure loss during transmission, thereby enhancing the suction applied to a human breast; the tee joint component is very convenient to clean, disassemble and assemble; at the same time, the sanitary conditions of the milking passage are improved, and the purity of milk is ensured.

The tee joint component is provided with a trumpet-shaped milking end which fits with the breast of a human body, and the thickness distribution of the tee joint component is non-uniform such that the middle region is thinner than other regions. Accordingly, the middle region of the milking end is liable to deform when the milking end of the tee joint component is under the negative pressure action. Since the tee joint component made of flexible materials, the tee joint component is easy to embed in a support. In order to facilitate cleaning and assembly, the support is mounted to a tee joint adapter, and internal threads are formed at the lower part of the tee joint adapter for the connection with a bottle. An air bag is arranged at the negative pressure input end of the tee joint component and equipped with an air bag cover, and the negative pressure input end of the tee joint component is connected with a negative pressure source through a straight-through joint on the air bag cover.

During the milking process, the parts exposed to milk are all made of food-grade silicone rubber, thereby making milk pure to the most extent. During the milking process, the effects of taking suction-massage, squeezing mammary areolas and stimulating lactation can be achieved. The milking shield and a valve support are integrally molded to make disassembly and assembly more convenient.

### BRIEF DESCRIPTION OF THE DRAWINGS

FIG. 1 is a sectional view of a tee joint component of the present invention.

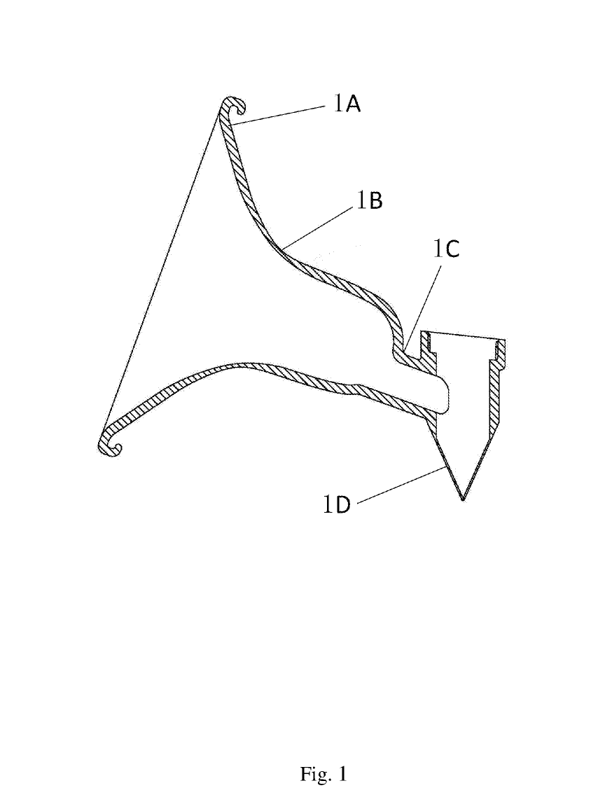

FIG. 2 is a sectional view of an electrical breast pump with the tee joint component of the present invention.

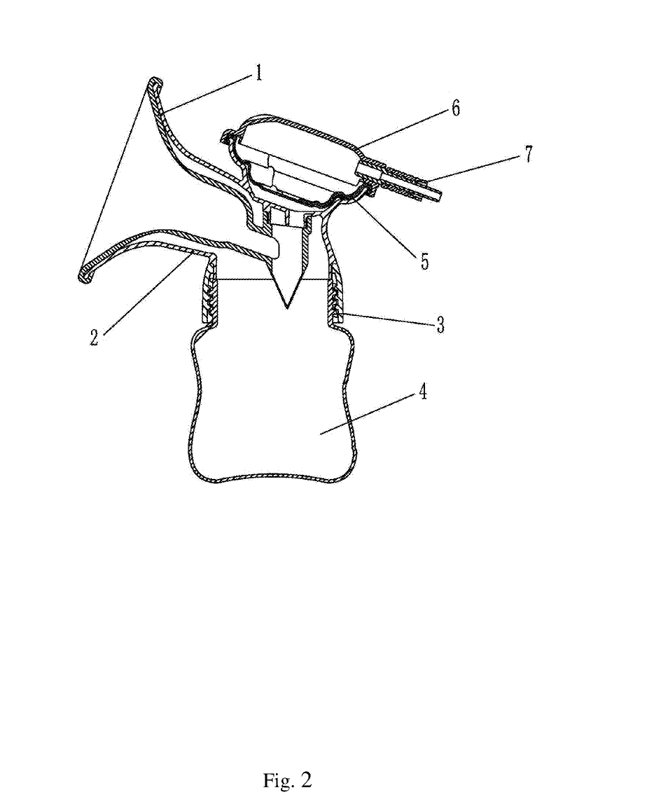

FIG. 3 is an exploded schematic diagram of an electrical breast pump with the tee joint component of the present invention.

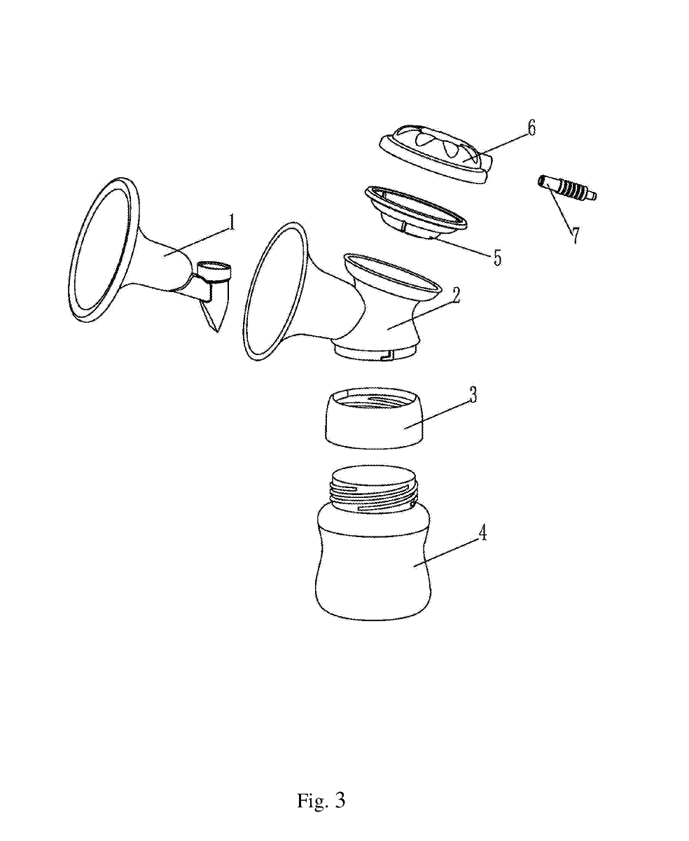

FIG. 4 is an exploded schematic diagram of a manual breast pump with the tee joint component of the present invention.

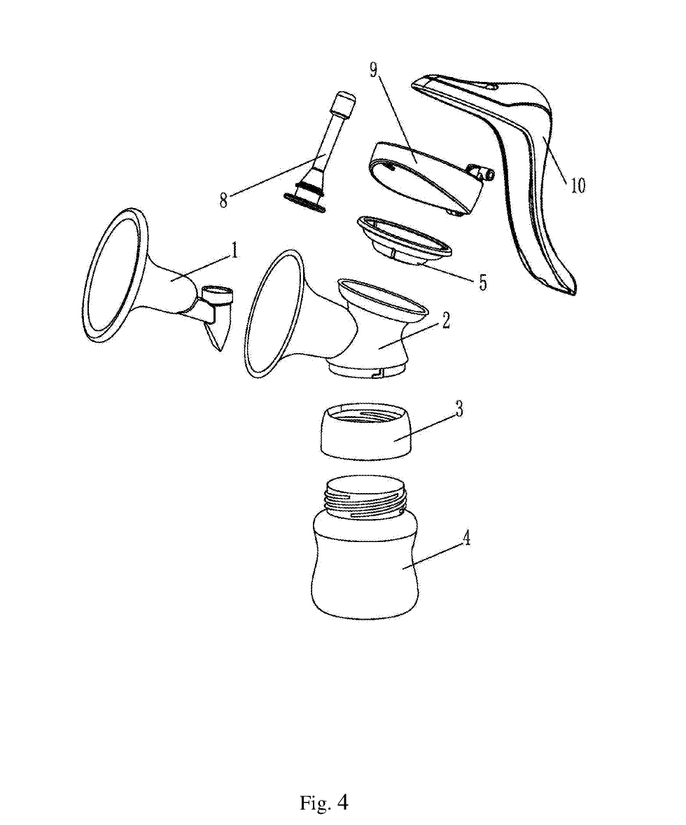

### SYMBOLS OF THE DRAWINGS

1-tee joint component, 1A-first region, 2A-second region, 3A-third region, 4A-fourth region, 2-support, 3-tee joint adapter, 4-bottle, 5-air bag, 6-air bag cover, 7-straight-through joint, 8-drawbar, 9-support cover, 10-handle.

### DETAILED DESCRIPTION OF THE INVENTION

The following further describes the present invention in detail in conjunction with drawings and embodiments.

### Embodiment 1

A tee joint component is provided. Unusually, the tee joint component is provided with only one negative pressure input interface; a milking shield and a milk outflow end which are arranged on the tee joint component are both integrated with the body of the tee joint component; the milk outflow end is a V-shaped one-way valve; the whole tee joint component is made of flexible materials, and is characterized in that the tee joint component is provided with only one negative pressure input interface; the milking shield and the milk outflow end which are arranged on the tee joint component are both integrated with the body of tee joint component; the milk outflow end is a V-shaped one-way valve; the whole tee joint component is made of flexible materials.

The cross-sectional thickness of both ends is larger than that of the middle region of the tee joint component; specifically, the cross-sectional thickness of the first region and the third region is greater than that of the second region and the fourth region.

The flexible material is food-grade silicone rubber.

### Embodiment 2

A breast pump is provided, comprising a negative pressure pump and a bottle. Unusually, the breast pump also comprises the tee joint component of Embodiment 1, connected with the negative pressure pump and the bottle.

The tee joint component is embedded in a support.

The support is connected with a tee joint adapter.

The tee joint adapter is connected with the bottle.

A negative pressure is outputted from a host machine diaphragm pump, and the negative pressure is fed to a tee joint shaped sucker. The negative pressure acts on the tee joint component in the tee joint shaped sucker, so that the tee joint component provides the effects of taking suction-massage, squeezing mammary areolas, and stimulating lactation and suction. Then the negative pressure is released by a solenoid valve in a host machine, so that the milk obtained by the tee joint component is fed into the bottle through the valve seat of the tee joint component. By the cyclic action in this manner, the whole process of lactation stimulation and milk suction is completed.

What is claimed is:

1. A tee joint component, characterized in that the tee joint component is only provided with one negative pressure input interface; a milking shield and a milk outflow end disposed on the tee joint component are both integrated with the body of the tee joint component; the milk outflow end is a V-shaped one-way valve; and the whole tee joint component is made of a flexible material.

2. The tee joint component of claim 1, characterized in that the cross-sectional thickness of both ends is greater than that of the middle region of the tee joint component; specifically, the cross-sectional thickness of the first region and the third region is greater than that of the second region and the fourth region.

3. The tee joint component of claim 1, characterized in that the flexible material is food-grade silicone rubber.

4. A breast pump comprising a negative pressure pump and a bottle, characterized by further comprising a tee joint component of claim 1 connected with the negative pressure pump and the bottle.

5. The breast pump of claim 4, the tee joint component is embedded in a support.

6. The breast pump of claim 5, characterized in that the support is connected with a tee joint adapter.

7. The breast pump of claim 6, characterized in that the tee joint adapter is connected with the bottle.
